(19) United States
(12) Patent Application Publication
(10) Pub. No.: US 2013/0030550 A1
(43) Pub. Date: Jan. 31, 2013

(54) MODULAR HUMAN HAND PROSTHESIS WITH MODULAR, MECHANICALLY INDEPENDENT FINGER MODULES

(71) Applicants: Wojciech Jopek, Zgorzelec (PL); Michal Turow, Wroclaw (PL); Michal Wasielewski, Legionowo (PL); Michal Kucharski, Jozefoslaw (PL)

(72) Inventors: Wojciech Jopek, Zgorzelec (PL); Michal Turow, Wroclaw (PL); Michal Wasielewski, Legionowo (PL); Michal Kucharski, Jozefoslaw (PL)

(21) Appl. No.: 13/522,167

(22) PCT Filed: Dec. 23, 2010
(86) PCT No.: PCT/PL2010/000124

§ 371 Date: Jul. 13, 2012

(30) Foreign Application Priority Data
Jan. 14, 2010 (PL) P-390188

(51) Int. Cl. A61F 2/54

(52) U.S. Cl. 623/64

(57) ABSTRACT

The invention is a modular human hand prosthesis with modular, mechanically independent finger modules, which may be applied in prosthetics as a replacement of a single finger, a group of fingers or whole hand or arm, as well as in robotics as a gripper. The device has at least one mechanically independent finger module, particularly consisting of a fixed finger base (8), a finger proximal segment (5), a finger distant segment (6), a rod 7 and a rotational drive (11); the finger base 8 is equipped with a rod rotational proximal joint (1) and the rotational joint of the finger proximal segment (2) is connected via the rod proximal joint (1), rod (7), the rod distant segment (4) and at the same time via the proximal joint of the finger proximal segment (2), the finger proximal segment (5), the distant joint of the finger proximal segment (3) with the finger distant segment (6), and at the same time the distant joint of the finger proximal segment (3) constitutes the rotational permanent centre of the drive (11).

The invention is a modular human hand prosthesis with modular, mechanically independent finger modules, which may be applied in prosthetics as a replacement of a single finger, a group of fingers or a whole hand or arm, as well as in robotics as a gripper.

Hand prosthesis, known from the Polish patent application no. P.372638, consists of a shaft axis with grasping fingers, which is twisted relative to the shaft axis with a prehensile thumb, and the thumb is rotary embedded with a change of twist angle in the yoke, while the shafts with fingers are connected with each other by means of a spherical toothed gear, and the drive shaft is connected by the conical toothed gear with a shaft with grasping fingers, and with the yoke by means of a cylindrical gear with a yoke.

Hand prosthesis, known from Polish patent application no. P.372639, consists of a shaft axis with grasping fingers, which is twisted to the shaft axis with prehensile thumb, and the thumb is rotary embedded with the change of twist angle in the yoke, while the shafts with fingers are connected with each other by means of a rotating link and a connector ended with ball-and-socket joints forming a spatial pentagon joint. At the same time, the yoke is connected via a cylindrical toothed gear with drive shaft, the shaft with gripping fingers through a conical toothed gear with the drive shaft, and the shaft along with the prehensile finger is connected with the drive shaft via a rotating element and a conical toothed gear.

The hand prosthesis, known from the Polish patent application no. P.379607, consists of a shaft axis with grasping fingers, which is twisted relative to the shaft axis with a prehensile finger embedded by means of a swivel with movement lock in the yoke, which enables a change of the twist angle. Shafts with fingers perform four types of movements because they are interconnected by a separable cylindrical toothed gear controlled by a disengaging clutch and two permanent conical toothed gears. The drive shaft is connected to the intermediate shaft with two disengaging cylindrical toothed gears controlled by disengaging clutches.

The finger prosthesis of the upper limb, known from the Polish patent application No. PRN/WI/451-12/09, has a linear drive, fixed at the finger base, equipped with an articulated joint connected through the first rod, the second articulated joint, the proximal phalanx and the fifth articulated joint with a distal phalange and, at the same time, the second joint is connected rigidly to the proximal phalanx rod bearing and rotatably with the third articulated joint, which is positioned at the finger base bearing. The fifth articulated joint is connected through the distant phalanx rod, the sixth joint and second rod with the fourth joint mounted at the finger base. The joints are located in the articulated pentagon corners performing movement resembling the movement of a finger.

The prosthesis, known from the patent application No. WO 9524875, consists of at least one mechanically efficient finger segment, with at least one finger segment positioned tangentially to a worm wheel fixed in the body of the prosthesis and at the same time bearinged rotationally in the worm wheel axis. The finger segment has a driving engine with a worm located along the finger segment and simultaneously coupled by means of the worm teeth to the worm wheel. While using the prosthesis, the finger segment moves around the worm wheel to or from another finger segment and/or to the natural finger opening in order to close and open the hand grip during the drive motor operation.

The prosthesis, known from the patent application No. WO 2007063266, consists of at least one mechanically efficient finger segment, which is positioned tangentially to a worm wheel fixed in the bracket segment of the prosthesis and at the same time bearinged rotationally in the worm wheel axis. The finger segment has a drive motor used to drive the worm. The worm is coupled with the worm wheel in a way that when the engine is turned on, the prosthesis is used, the finger segment moves around the worm wheel, which is located outside the finger segment.

At the moment, the development of a prosthesis allowing to grasp objects of different shapes, sizes and dimensions and weights with adjustable strength is a considerable problem. An additional requirement, considering the support for independent movement of individual fingers in a manner similar to the natural movement of the fingers, while keeping the weight and dimensions of the device similar to those typical of the human hand, makes this task even more complicated.

Another problem is the development of a prosthesis which will have a modular design characterized by independent mechanical fingers of at least one independent and one dependent degree of freedom, and of a drive system located inside the finger in such a way as to allow the device to replace individual lost fingers as well as an entire arm and/or hand.

The most popular solution on the market for hand prosthetics generally does not have a modular design and only enables a simple hand opening and closing movement and wrist rotation of the hand. The conventional approach is characterized by using a single drive system that drives all the fingers simultaneously. The drive systems of other solutions that are available, whose authors decided to use independently driven fingers, have been developed in such a way that a part of the system together with the motor is located inside a finger. However, unfortunately, the rest of the system is located within the metacarpus. Consequently, it limits the possibility of using the device in case of the absence of a single finger or a group of fingers.

The invention of the modular hand prosthesis described below, with modular, mechanically independent finger modules, solves at least some of the aforementioned problems by placing the entire single finger drive system inside the finger and using the additional space available in the distant phalanx of the prosthesis.

The essence of the invention lies in the fact that it has at least one mechanically independent finger module which in particular consists of a fixed base, a finger proximal segment, a finger distant segment, a rod and a rotational drive, while the finger base equipped with a rotating proximal joint of the rod and the rotating proximal joint of the finger proximal segment is connected via the rod proximal joint, the rod, the rod distant joint and, simultaneously, via the proximal joint of the finger proximal segment, the finger proximal segment, the distant joint of the finger proximal segment with the finger distant segment, and the distant joint of the finger proximal segment constitutes the permanent centre of the rotational drive.

A finger is composed of at least two segments, of which the finger proximal segment is applied as the proximal phalanx and the finger distant segment is used as the distant phalanx.

Preferably, in particular the electric motor permanently coupled with reduction gear, which is connected with the worm gear consisting of a worm and worm wheel, is used as a rotational drive. The electric motor together with the reduction gear is fixed in the finger proximal segment in such a way that the reduction gear output shaft is facing the finger distant segment. hi turn, the worm wheel is fixed to the finger distant segment, while the worm, which is geared with it, is mounted on the reduction gear output shaft. The electric motor with a reduction gear is located along the proximal section of the finger while the motor axis is inclined from the geometrical axis of the finger proximal segment in a range of ±30°.

Preferably, when the motor is supplied with power, the worm mounted on the engine output shaft rotates the worm wheel and the associated finger distant segment against the finger proximal segment and particularly against the distant joint of the finger proximal segment.

Preferably, when the finger distant segment moves, it causes rotational movement of the distant segment of the proximal finger in relation to the joint of the distant rod and the rod in relation to the joint of the proximal rod, causing rotational movement of the finger proximal segment in relation to the proximal segment joint of the of the proximal finger of the finger base.

Preferably, when the motor is powered, the finger proximal segment and the finger distant segment perform, relative to the finger base, a complex movement in such a way that the finger distant segment approaches or moves away from the finger base and the finger proximal segment performs a rotational movement relative to the proximal joint of the finger proximal segment of the finger base, causing the closing or opening of the finger module.

Preferably, the rotational drive's function is performed by the electric, pneumatic or hydraulic motor which, through a single gear, a set of gears or without a gear, enables the movement of the finger module.

Preferably, the mechanical system causing the finger distant segment rotational movement is in particular the mechanical and/or hydraulic and/or pneumatic gear.

Preferably, the ratio of the horizontal part distance of the finger distant segment comprising the distant joint of the finger proximal segment and the rod distant joint to the part perpendicular to it is 0.24 a:a, where a is the dimension characteristic for the finger distant segment while the ratio of distance between the distant joint of the finger proximal segment and the rod distant joint to the part of the finger distant segment perpendicular to the horizontal part containing the distant joint of the finger proximal segment and the rod distant joint is 0.07 a.

Preferably, the lines formed by the rod proximal joint and the proximal joint of the finger proximal segment and the distant joint of the finger proximal segment and the rod distant joint are parallel to one another if the mechanically independent finger module is in its initial position.

Preferably, the ratio of the distance between the rod proximal joint and the proximal joint of the finger proximal segment to the vertical distance between the proximal joint of the finger proximal segment and the rod distant joint is 0.32 b:b, where b is the characteristic dimension for the finger proximal segment.

Preferably, the ratio of the horizontal distance of the proximal joint of the finger proximal segment from the finger distant segment part perpendicular to the horizontal part containing the distant joint of the finger proximal segment and the rod distant joint, to the vertical distance between the proximal joint of the finger proximal segment and the rod distant joint is 0.1 b:b.

Preferably, the ratio of the characteristic dimension ‘a’ of the finger distant segment to the characteristic dimension ‘b’ of the finger proximal segment is 0.9.

Preferably, according to the invention, the device has a single, mechanically independent finger module together with elements connecting the finger module basis with the patient's body, replacing a patient's single lost finger.

Preferably, according to the invention, there are up to four mechanically independent finger modules with the load bearing structure and/or structures connecting the base of all modules or groups of modules, replacing the lost fingers group.

Preferably, according to the invention, the device has five mechanically independent finger modules with the load bearing structure connecting the bases of all modules, and forms a structure that replaces all the lost fingers with or without replacing the hand.

Preferably, according to the invention, there are additional components and/or load bearing structures especially in the form of wrist connectors, wrist, fastening systems in case of upper limb short amputation, belts, harnesses, and other elements performing the cosmetic and/or protective function in relation to the mentioned additional components and/or load bearing.

Preferably, the device is controlled by digital and/or analog processed electromyography signals (EMG) and/or mechanomyographic signals (MMG), in particular acousticmyographic and/or accelleromyographic signals, and other biological signals including in particular electrooculographic (EOG) and electroencepbalographic (EEG) signals.

Preferably, according to the invention, the device has an outer shell and/or layers that perform an aesthetic and/or protective function against external factors.

The device has a modular design based on fitting a fully functional drive system inside a single finger module, making it fully mechanically independent. The kinematic structure and the type of mechanical gear used mean that the size of the device and its weight depends almost exclusively on the desired strength generated by a grasping finger. In addition, a small number of elements in the kinematic chain of a single mechanically independent finger module significantly increase the device reliability and technological complexity.

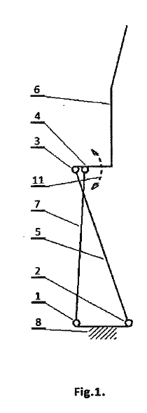

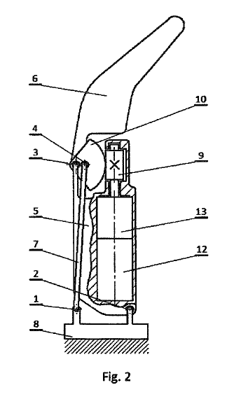

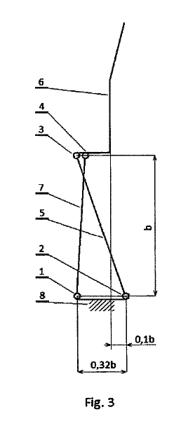

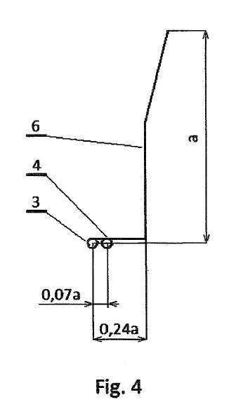

The invention in the form of a sample is shown in the drawing, in which FIG. 1 shows the kinematic diagram of the mechanically independent finger module, FIG. 2—a sample of the mechanically independent finger module, FIG. 3, a kinematic diagram of the mechanically independent finger module with marked geometrical dependence describing the mutual position of the finger distant segment 6 relative to the finger proximal segment 5 and the values characterizing the finger proximal segment 5 and the finger basis 8, FIG. 4 a kinematic diagram of the mechanically independent finger module with selected geometrical dependences describing the finger distant segment 6.

### EXAMPLE I

The modular human hand prosthesis with modular, mechanically independent finger modules, has a single mechanically independent finger module together with elements connecting the base 8 of the finger module with the patient's body, which replaces a single lost finger of the patient, consisting of, in particular, a fixed finger base 8, the finger proximal segment 5, the finger distant segment 6, rod 7 and the rotational drive 11, wherein the finger base 8, equipped with rod rotational proximal joint 1 and the rotational joint of the finger proximal segment 2, is connected via the rod proximal joint 1, rod 7, the rod distant segment 4 and simultaneously via the proximal joint of the finger proximal segment 2, the finger proximal segment 5, the distant joint of the finger proximal segment 3 with the finger distant segment 6, wherein the distant joint of the finger proximal segment 3 is the rotational permanent centre of the drive 11. The finger proximal segment 5 is used as the proximal phalanx and the finger distant segment 6—as the distant phalanx. The rotational drive 11 is provided by the electrical motor 12 coupled permanently with reduction gear 13, which is connected to worm gear 9, 10. In the finger proximal segment 5, the electric motor 12 is fixed together with reduction gear 13 in such a way that the reduction gear output shaft is facing the finger distant segment 6. Worm wheel 10, which is connected permanently with the finger distant segment 6, is geared with worm 9, mounted on the reduction gear output shaft 13. The electric motor 12, together with the reduction gear 13, is located along the finger proximal segment 5 and the motor axis 13 is inclined from the geometrical axis with the axis of the finger proximal segment 5 in the range of ±30°. The ratio of the horizontal part distance of the finger distant segment 6 comprising joints 3 and 4 to the part perpendicular to it is 0.24 a:a, where a is the dimension characteristic of the finger distant segment 6 while the ratio of distance between joints 3 and 4 to the part of the finger distant segment 6 perpendicular to the horizontal part containing joints 3 and 4 is 0.07 a. The lines formed by joints 1 and 2 as well as joints 3 and 4 are parallel to each another if the mechanically independent finger module is in its initial position. The ratio of the distance between joints 1 and 2 to the vertical distance between joints 2 and 4 is 0.32 b:b, where b is the dimension characteristic of the finger proximal segment 5 while the ratio of the horizontal distance of joint 2 from the part of the finger distant segment 6 perpendicular to the horizontal part containing joints 3 and 4 to the vertical distance between joints 2 and 4 is 0.1 b:b. The ratio of the characteristic dimension ‘a’ of the finger distant segment 6 to the characteristic dimension ‘b’ of the finger proximal segment 5 is 0.9.

The device offers the possibility to use additional construction components and/or load bearing especially in the form of wrist connectors, wrist, fastening systems in case of upper limb short amputation, belts, harnesses, and other elements serving as cosmetic and/or protective function in relation to the mentioned additional components and/or load bearing. Moreover, the prosthesis as invented is controlled by digital and/or analog processed electromyography signals (EMG) and/or mechanomyographic signals (MMG), in particular acousticmyographic and/or accelleromyographic signals, and other biological signals including in particular electrooculographic (EOG) and electroencephalographic (EEG) signals. The device may also be equipped with an outer shell and/or having as an aesthetic and/or protective function against external factors.

Electric motor 12 is responsible for forcing the movement of the mechanically independent prosthesis finger module. When powered, worm 9 mounted on the motor output shaft 12 causes worm wheel rotation 10 and the related finger distant segment 6 in relation to the finger proximal segment 5 and in particular in relation to the distant joint of the finger proximal segment 3. Then, when the finger distant segment 6 moves, the rotational movement of the distant joint of the finger proximal segment 3 against the rod distant joint 4 and rod 7 in relation to the rod proximal joint 1 takes place, causing rotational movement of the finger proximal segment 5 in relation to the proximal joint of the finger proximal segment 2 of the finger base 8. Therefore, when the motor 12 is powered, the finger proximal segment 5 and the finger distant segment 6 perform a complex movement against the finger base 8 in such a way that the finger distant segment 6 approaches or moves away from the finger base 8 and from the finger proximal segment 5, and the proximal segment performs rotational movement relative to the proximal joint of the finger proximal segment 2 of the finger base 8 causing the finger module to open or close.

### EXAMPLE 2

The modular human hand prosthesis with modular, mechanically independent finger modules as in example 1 except that the role of the rotational drive 11 is performed by an electrical, pneumatic or hydraulic motor which, through a single gear, set of gears or without any gear, enables the movement of a finger module.

### EXAMPLE 3

The modular human hand prosthesis with modular, mechanically independent finger modules as in example 1 except that it is the mechanical and/or hydraulic and/or pneumatic gear that causes the rotational movement of the finger distant segment 6 against the finger proximal segment 5.

### EXAMPLE 4

The modular human hand prosthesis with modular, mechanically independent finger modules as in the example 1 with the exception that it has from 2 to 4 mechanically independent finger modules with a load bearing structure and/or structures connecting the bases 8 of all modules or groups of modules, replacing the lost fingers group.

### EXAMPLE 5

The modular human hand prosthesis with modular, mechanically independent finger modules as in the example 2, substantial in that it has from 2 to 4 mechanically independent finger modules with the load bearing structure and/or structures connecting the bases 8 of all modules or groups of modules, replacing the lost fingers group.

### EXAMPLE 6

The modular human hand prosthesis with modular, mechanically independent finger modules as in the example 3 substantial in that it has from 2 to 4 mechanically independent finger modules with the load bearing structure and/or structures connecting the bases 8 of all modules or groups of modules, replacing the lost fingers group.

### EXAMPLE 7

The modular human hand prosthesis with modular, mechanically independent finger modules as in the example 1 substantial in that it has 5 mechanically independent finger modules with the load bearing structure connecting the bases 8 of all modules forms a design replacing all the lost hand fingers with or without the need to replace the whole hand.

### EXAMPLE 8

The modular human hand prosthesis with modular, mechanically independent finger modules as in the example 2 substantial in that it has 5 mechanically independent finger modules with the load bearing structure connecting the bases 8 of all modules forms a design replacing all the lost hand fingers with or without the need to replace the whole hand.

### EXAMPLE 9

The modular human hand prosthesis with modular, mechanically independent finger modules as in the example 3 substantial in that it has 5 mechanically independent finger modules with the load bearing structure connecting the bases 8 of all modules forms a design replacing all the lost hand fingers with or without the need to replace the whole hand.

1. The modular human hand prosthesis with modular, mechanically independent finger modules which comprises at least one mechanically independent finger module comprising a fixed finger base (8), a finger proximal segment (5), a finger distant segment (6), a rod (7) and a rotational drive (11), wherein the finger base (8), equipped with a rod rotational proximal joint (1) and a rotational joint (2) of the finger proximal segment (5), is connected via the rod proximal joint (1), rod (7), a rod distant joint (4) and simultaneously via the proximal joint (1) of the finger proximal segment (2), the finger proximal segment (5), the distant joint of the finger proximal segment (3) with the finger distant segment (6), wherein the distant joint of the finger proximal segment (3) constitutes the rotational centre of the drive (11).

2. The modular human hand prosthesis hand with modular, mechanically independent finger modules according to claim 1, wherein the at least one mechanically independent finger is composed of at least two segments, of which the finger proximal segment (5) is the proximal phalanx and the finger distant segment (6) is the distant phalanx.

3. The modular human hand prosthesis with modular, mechanically independent finger modules according to claim 1, wherein electric motor (12), coupled permanently to the reduction gear (13), which is connected to worm gear (9,10), is used as the main rotational drive (11).

4. The modular human hand prosthesis with modular, mechanically independent finger modules according to claim 3, wherein the electric motor (12) together with reduction gear (13) is permanently fixed in the finger proximal segment (5) in such a way that the reduction gear output shaft is facing the finger distant segment (6).

5. The modular human hand prosthesis with modular, mechanically independent finger modules according to claim 4, wherein the worm wheel (10) is fixed to the finger distant segment (6).

6. The modular human hand prosthesis with modular, mechanically independent finger modules according to claim 4, wherein the worm gear (9) geared with worm wheel (10) is mounted on the output shaft of the reduction gear (13).

7. The modular human hand prosthesis with modular, mechanically independent finger modules according to claim 4, wherein the electric motor (12) together with the reduction gear (13) is positioned along the finger proximal segment (5), wherein the axis of the motor (13) is inclined from the geometrical axis of the finger proximal segment (5) in the range of ±30°.

8. The modular human hand prosthesis with modular, mechanically independent finger modules according to claim 3, wherein at the time the motor (12) is powered, the worm gear (9) mounted on the motor output shaft of the motor (12), causes rotation of worm wheel (10) and the associated finger distant phalanx (6) against the finger proximal phalanx (5) and, in particular, against the distant joint (3) of the finger proximal phalanx (5).

9. The modular human hand prosthesis with modular, mechanically independent finger modules according to claim 1, wherein when the finger distant segment (6) moves it causes rotational movement of the distant joint (3) of the finger proximal segment (5) in relation to the rod distant joint (4) and rod (7) in relation to the rod proximal joint (1), causing rotational movement of the finger proximal segment (5) in relation to the rod proximal joint (2) of the finger proximal segment (5) of the finger base (8).

10. The modular human hand prosthesis with modular, mechanically independent finger modules according to claim 3, wherein when the motor (12) is powered, the finger proximal segment (5) and the finger distant segment (6) perform, relative to the finger base (8), a complex movement in such a way that the finger distant segment (6) approaches or moves away from the finger base (8) and the finger proximal segment (5), while the proximal segment performs a rotational movement relative to the proximal joint (2) of the finger proximal segment (5) of the finger base (8), causing the closing or opening of the finger module.

11. The modular human hand prosthesis with modular, mechanically independent finger modules according to claim 1, wherein the rotational drive (11) is provided by electric, pneumatic or hydraulic motor which, through a single gear, a set of gears or without any gear enables the movement of a finger module.

12. The modular human hand prosthesis with modular, mechanically independent finger modules according to claim 1, wherein it is the mechanical and/or hydraulic and/or pneumatic gear that causes rotational movement of the finger distant segment (6) against the finger proximal segment (5).

13. The modular human hand prosthesis with modular, mechanically independent finger modules according to claim 1, wherein the ratio of the distance of the horizontal part of the finger distant segment (6) comprising joints (3) and (4) against the perpendicular part to it is 0.24 a:a, where ‘a’ is a characteristic dimension of the finger distant segment (6).

14. The modular human hand prosthesis with modular, mechanically independent finger modules according to claim 1, wherein the ratio of the distance between joints (3) and (4) to the part of the distant segment (6) perpendicular to the horizontal part containing joints (3) and (4) is 0.07 a.

15. The modular human hand prosthesis with modular, mechanically independent finger modules according to claim 1, wherein the lines formed by joints (1) and (2), as well as joints (3) and (4), are parallel to each another if the mechanically independent finger module is in its initial position.

16. The modular human hand prosthesis with modular, mechanically independent finger modules according to claim 15, wherein the ratio of the distances between joints (1) and (2) to the vertical distance between joints (2) and (4) is 0.32 b:b, where ‘b’ is a characteristic dimension of the finger proximal segment (5).

17. The modular human hand prosthesis with modular, mechanically independent finger modules according to claim 15, wherein the ratio of the horizontal distance of joint (2) from the part of the finger distant segment (6) perpendicular to the horizontal part containing joints (3) and (4) to the vertical distance between joints (2) and (4) is 0.1 b:b.

18. The modular human hand prosthesis with modular, mechanically independent finger modules according to claim 1, wherein the ratio of the characteristic dimension ‘a’ of the finger distant segment (6) to the characteristic dimension ‘b’ of the finger proximal segment (5) is 0.9.

19. The modular human hand prosthesis with modular, mechanically independent finger modules according to claim 1, which comprises a single mechanically independent finger module with an element connecting the finger module base (8) with the patient's body and replaces the patient's single lost finger.

20. The modular human hand prosthesis with modular, mechanically independent finger modules according to claim 1, which comprises from 2 to 4 or 5 mechanically independent finger modules with the load bearing structure and/or structures connecting the bases (8) of all modules or groups of modules, replacing the lost fingers group.

21-24. (canceled)
